(19) United States
(12) Patent Application Publication
(10) Pub. No.: US 2026/0007314 A1
(43) Pub. Date: Jan. 8, 2026

(54) MINIATURE OPTICAL DEVICE FOR MONITORING LOCAL PULSE WAVE VELOCITY

(71) Applicant: Medtronic, Inc., Minneapolis, MN (US)

(72) Inventors: Jason C. Lee, Edina, MN (US); Michael B. Terry, Seattle, WA (US)

(21) Appl. No.: 18/994,181

(22) PCT Filed: Jul. 27, 2023
(86) PCT No.: PCT/US2023/071122

§ 371 Date: Jan. 14, 2025

Related U.S. Application Data
(60) Provisional application No. 63/369,873, filed on Jul. 29, 2022.

(51) Int. Cl. A61B 5/02; A61B 5/021

(52) U.S. Cl. CPC A61B 5/02007; A61B 5/02125

(57) ABSTRACT

Devices, systems and techniques to measure changes in pulse transit time (PTT) and, in some cases, determine pulse wave velocity (PWV), in a blood vessel to support continuous ambulatory monitoring of PTT and/or PWV. Each heartbeat creates a pressure wave that propagates along the arterial system. A pressure wave may travel faster along a rigid artery when compared to a more flexible artery. In this manner, PTT may be an indirect indicator of blood vessel flexibility and patient health.

This Application claims the benefit of U.S. Provisional Patent Application 63/369,873 filed Jul. 29, 2022, the entire content of which is incorporated herein by reference.

### TECHNICAL FIELD

The disclosure relates measuring pulse transit time between two points in a blood vessel after a contraction of the cardiac heart muscle.

### BACKGROUND

One factor in patient health may include blood vessel wall flexibility. In some examples, blood vessels may become chronically more rigid over time because of age-related issues, arteriosclerosis and other factors. Blood vessels may also acutely change rigidity, e.g., in the short term, based on for example, control by the autonomic nervous system.

### SUMMARY

In general, the disclosure describes devices, systems and techniques to determine changes in pulse transit time (PTT) in a blood vessel of a patient and to support continuous ambulatory monitoring of PTT. Each heartbeat creates a pressure wave that propagates along the arterial system. The velocity of this wave is referred to as pulse wave velocity (PWV). The inverse of PWV is called pulse transit time (PTT). This disclosure describes techniques to measure PTT of the propagated wave using one or more optical sensors and, in some examples, calculate PWV. The miniature size of the device of this disclosure used to measure PTT may be implantable in the patient to support chronic and continuous monitoring for changes in PTT.

The device may contain one or two photo-emitters, two photo-detectors, and a high-resolution temporal (timer) circuit. In contrast to other systems that measure a time the pressure wave takes to travel over a longer distance, e.g., approximately one meter of artery, the apparatus of this disclosure may measure the time required to travel over a short section of an artery. For example, the monitored length of the artery may be approximately 50 mm, 40 mm, 10 mm or shorter. Because of the short time required for the pressure wave to travel through a short distance, a circuit with a high temporal resolution may be desirable, e.g., to detect small changes in the travel time for the pressure wave, such as changes on the order of 100 microseconds (μs) or less.

PTT may provide information on the rigidity of an artery because PWV (and thus PTT) may vary based on arterial wall rigidity and may therefore be an indicator of one risk factor for cardiovascular disease. High pressure at one end of a rigid blood vessel may be sensed quickly at the other end. The pressure wave may travel less quickly through a more flexible, less rigid blood vessel. Because the apparatus and methods of this disclosure enable measurement of PTT along a relatively shorter length of vessel, the apparatus of this disclosure can be miniaturized, and may be implemented as an implantable device, a wearable device, or a highly portable instrument. A device based on this disclosure may monitor PWV at any site near an artery.

A device or system implementing the techniques of this disclosure may provide clinical benefits, such long-term monitoring of PWV with little patient inconvenience and therefore likely improved patient compliance, when compared to other PWV monitoring. The small size may also allow the PTT measurement device to be included with one or more other wearable or implantable functions, e.g., glucose monitoring, cardiac monitoring and similar functions. Chronic monitoring may also provide the clinical benefit of trend analysis and early warning of possible changes in the patient for preemptive care before the patient shows other symptoms.

In one example, this disclosure describes a medical system comprising: a medical device including: at least one optical emitter configured to output light to tissue of a patient; a first optical detector and a second optical detector configured to receive the light reflected from the tissue of the patient; processing circuitry configured to: receive an indication from the first optical detector of a pressure wave from a cardiac contraction passing the first optical detector; receive an indication from the second optical detector of the pressure wave from the cardiac contraction passing the second optical detector; determine a pulse transit time of the pressure wave between the first optical detector and the second optical detector based on: the received indication from the first optical detector, and the received indication from the second optical detector; and output an electronic signal comprising a health indication for the patient based on the determined pulse transit time.

In another example, this disclosure describes a medical device comprising: at least one optical emitter configured to output light to tissue of a patient; a first optical detector and a second optical detector configured to receive the light reflected from the tissue of the patient; processing circuitry configured to: receive an indication from the first optical detector of a pressure wave from a cardiac contraction passing the first optical detector; receive an indication from the second optical detector of the pressure wave from the cardiac contraction passing the second optical detector; determine a pulse transit time of the pressure wave between the first optical detector and the second optical detector based on: the received indication from the first optical detector, and the received indication from the second optical detector; and output an electronic signal comprising a health indication for the patient based on the determined pulse transit time.

In another example, this disclosure describes a method comprising: controlling, by processing circuitry of a medical device, at least one optical emitter to output light to tissue of a patient; receiving, by the processing circuitry, an indication from a first optical detector of a pressure wave from a cardiac contraction passing the first optical detector; receiving, by the processing circuitry, an indication from a first optical detector of a pressure wave from a cardiac contraction passing the first optical detector, wherein the first optical detector and the second optical detector are configured to receive light reflected from the tissue of the patient; determining, by the processing circuitry, a pulse transit time of the pressure wave between the first optical detector and the second optical detector based on: the received indication from the first optical detector, and the received indication from the second optical detector; and outputting an electronic signal comprising a health indication for the patient based on the determined pulse transit time.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1A is a conceptual diagram illustrating a system including a device configured to measure pulse transit time according to one or more techniques of this disclosure.

FIG. 1B is a conceptual diagram illustrating an example medical device arranged in proximity to tissue of the patient.

FIG. 2A is a block diagram illustrating an example implementation of device configured to measure pulse transit time, which uses a single optical emitter.

FIGS. 2B and 2C are block diagrams illustrating example pulse transit time measuring devices with a single optical emitter.

FIG. 3A is a block diagram illustrating an example implementation of device configured to measure pulse transit time, which uses at least two optical emitters.

FIGS. 3B and 3C are block diagrams illustrating example pulse transit time measuring devices with two or more optical emitters.

FIG. 4 is a flow chart illustrating an example operation of the pulse transit time measurement system of this disclosure.

### DETAILED DESCRIPTION

The disclosure describes devices, systems and techniques to measure changes in pulse transit time (PTT) and, in some cases, determine pulse wave velocity (PWV), in a blood vessel to support continuous ambulatory monitoring of PTT and/or PWV. Each heartbeat creates a pressure wave that propagates along the arterial system. A pressure wave may travel faster along a rigid artery when compared to a more flexible artery. In this manner, PTT may be an indirect indicator of blood vessel flexibility.

In some examples, blood vessels may become chronically more rigid over time because of age-related issues, arteriosclerosis and other factors. Blood vessels may also acutely change rigidity, e.g., in the short term, based on for example, control by the autonomic nervous system. The autonomic nervous system has three branches: the sympathetic nervous system, the parasympathetic nervous system and the enteric nervous system. In some examples the autonomic may control blood pressure, for example, by stimulating the sympathetic system to constrict blood vessels or the parasympathetic system to relax blood vessels. Blood pressure control may be based on patient blood chemistry, posture, activity and other factors.

The device of this disclosure may have advantages over devices that measure PTT over a distance, e.g., from the carotid artery to the femoral artery. A miniaturized device of this disclosure (implantable, wearable, or portable) may measure the PTT and may provide a means to continuously monitor PWV, which may also simplify the ability to monitor PWV in a clinic or in an operating room, in addition to ambulatory measurements described above. Also, the device of this disclosure may need only access of one site instead of other techniques that use two sites that are far apart (e.g., 1 meter). In this disclosure, the term “miniaturized” may include a device limited to less than a few cubic centimeters (cc), e.g., five ccs, and configured to operate on a few hundred nano amperes (nA), which may be useful for an implantable device with an internal power supply, such as a battery, super capacitor or some similar energy storage device. In some examples, the miniature device may be less than 1.5 ccs and in other examples less than 1.2 ccs.

An implantable device may offer the long-term monitoring with little patient inconvenience and therefore excellent patient compliance. In addition, such monitoring capability can be combined with other sensing modalities for management of chronic diseases involving cardiovascular health. The disclosed circuit topology may be configured to detect a change in the arrival time difference of blood pulse wave of 100 μs or shorter.

The techniques of this disclosure may have advantages over devices that use synchronization with an electrocardiogram (ECG) to measure PTT. Some equipment to measure PTT may include measuring a time between an electrocardiogram R-wave to the arrival time of the pressure wave downstream in the artery. In some examples, such a device may use separate sensors to measure the PWV by measuring the time between an electrical sensor that measures an ECG R-wave to the arrival time of the pressure wave using an optical sensor. Such a synchronization approach (1) may use two different sensor modalities, (2) may have some timing uncertainty caused by the pre-ejection period, and (3) could be limited to measurement sites where an ECG can be reliably acquired. In contrast, the device of this disclosure may use the signals from two optical sensors to determine a time difference for the passage of a cardiac pressure wave in a blood vessel.

The device of this disclosure may also be a lower cost and portable alternative to other examples of devices that measure patient hemodynamics. Some other examples of devices may include multidimensional optical sensors configure to generate images within a field of view of the optical sensor. Such devices may be too large to be implantable, and such a sensor, and signal processing for a multidimensional sensor, may consume more power than a small, implantable battery could provide. In contrast, some examples of the device of this disclosure may be implanted under the skin of a patient with a simple procedure, provide long-term monitoring of patient hemodynamics and be unobtrusive with little to no impact on the daily life of the patient.

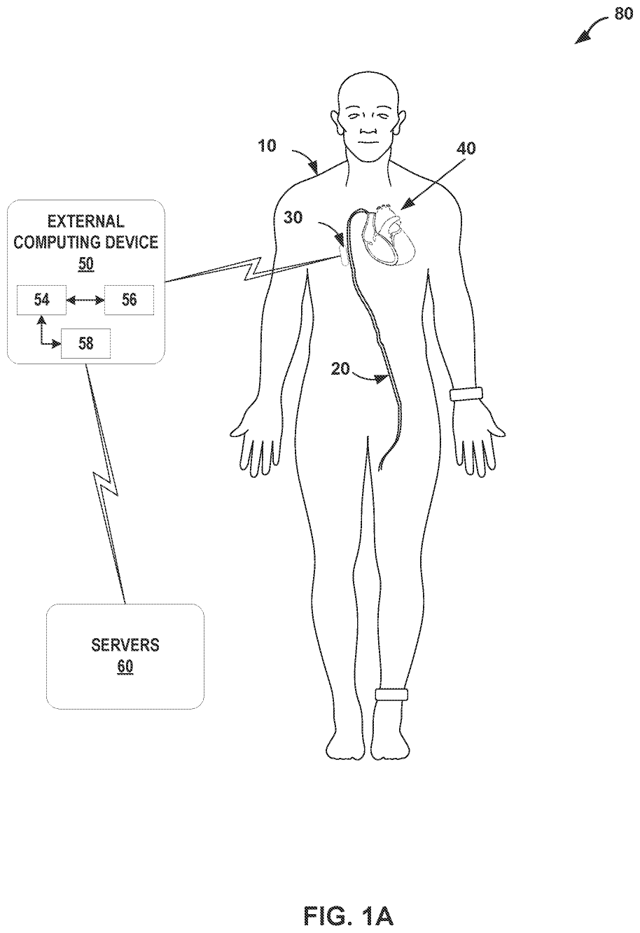

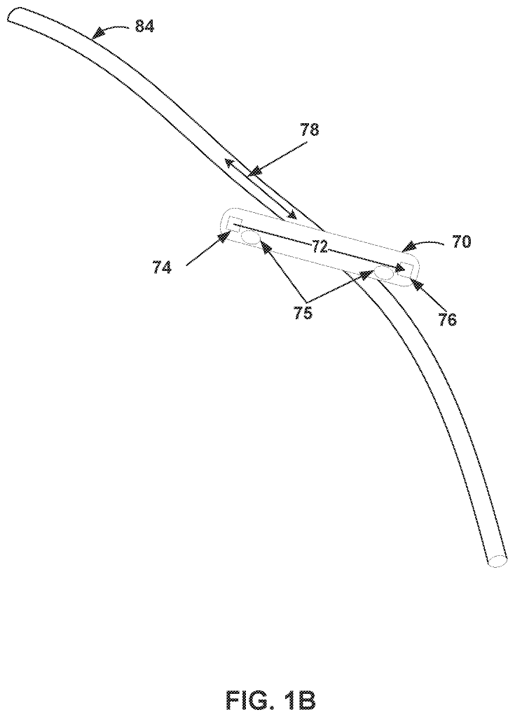

FIG. 1 is a conceptual diagram illustrating a system including a device configured to measure pulse transit time according to one or more techniques of this disclosure. System 80, in the example of FIG. 1 may include a medical device 30, which may be in communication with an external computing device 50 and servers 60. In some examples medical device 30 may be a wearable device, an implantable medical device (IMD) or a portable device.

In the example of an implantable medical device, IMD 30 may be implanted outside of a thoracic cavity of patient 4, e.g., subcutaneously in the pectoral location or other locations where a blood vessel e.g., 20, may be within a field of view of optical emitter and optical sensors of IMD 30. In other examples, IMD 30 may be positioned near the sternum or just below the level of heart 40, e.g., at least partially within the cardiac silhouette. In some examples, IMD 30 takes the form of a LINQ™ Insertable Cardiac Monitor (ICM), available from Medtronic, Inc.

In the example of a wearable device, medical device 30 may be worn in a belt, strap, finger clamp, vest or some other garment, or may be attached to a location of patient 10 with an adhesive, such that a blood vessel 20 of patient 10 may be within the field of view of the sensors of medical device 30. Similarly, in the example of a portable device, the sensors of medical device 30 may be placed on patient 10 to include any dominant blood vessel, e.g., an arteriole, in the field of light, where the longitudinal axis of the blood vessel does not run perpendicular to the vector defined by two photo detectors of medical device 30 (not shown in FIG. 1A).

External device 50 may be a computing device configured for use in locations such as a home, clinic, or hospital, and may further be configured to communicate with IMD 30 via wireless telemetry. For example, external device 50 may be coupled to a remote patient monitoring system hosted on servers 60, such as Carelink®, available from Medtronic. External device 50 may, in some examples, be implemented as a programmer, an external monitor, or a consumer device such as a smart phone or tablet. Processing circuitry 54 of external computing device 50 may operatively connect to memory 56. In some examples any of the functions described herein may be performed by processing circuitry of medical device 30 (not shown in FIG. 1A), by processing circuitry 54, or some combination of the processing circuitry of system 80, including processing circuitry of servers 60. Some examples of external computing device 50 may include wireless power transfer circuitry to charge a battery or similar electrical energy storage component that may power medical device 30.

External device 50 may program commands or operating parameters into medical device 30 for controlling its functioning (e.g., when configured as a programmer). In some examples, external device 50 may interrogate IMD 30 to retrieve data, including device operational data as well as physiological data accumulated in a memory of medical device 30 (not shown in FIG. 1A). Such interrogation may occur automatically according to a schedule or may occur in response to a remote or local user command. Programmers, external monitors, and consumer devices are examples of external device 50 that may be used to interrogate medical device 30. Examples of communication techniques used by medical device 30 and external device 50 include radiofrequency (RF) telemetry, which may be an RF link established via Bluetooth®, Near Field Communication (NFC), WiFi, or medical implant communication service (MICS).

In some examples, external device 50 may include a user interface 58 configured to allow a clinician to remotely interact with medical device 30. User interface 58 may include one or more displays, such as a display screen, indicator lights, and similar items configured to convey information to patient 10, a clinician or some other caregiver. For example, user interface 58 may receive an electronic signal that includes information about the health of patient 10 based on the measured PTT from processing circuitry 54 and may output an indication of the health of patient 10. The indication may take the form of a graphic, such as a traffic semaphore with red, yellow or green display to give a general health indication. The output may also include charts, graphs, text or other information to provide a more detailed indication of the health of patent 10 based on the measured PTT. User interface 58 may include one or more audio and/or haptic output components. User interface 58 may also include controls such as knobs, keys, buttons, levers, sliders and so on. In some examples e.g., a clinician may also remotely interact with medical device 30 via servers 60. Processing circuitry 54 may output the electronic signal that includes information about the health of patient 10 based on the measured PTT to servers 60, which may provide the patient 10 health information to a clinician at a location remote from patient 10.

The monitoring features of system 80 may provide advantages when compared to other types of monitoring systems. For examples, system 80 may collect data and track the health parameters of patient 10 with little or no action required of patient 10, as described above.

Medical device 30 may include a plurality of electrodes (not illustrated in FIG. 1A) and a set of sensors including one or more optical sensors, e.g., photo detectors (not illustrated in FIG. 1), which collectively detect signals that enable processing circuitry of system 80 to determine current values of at least one patient parameter associated with patient 10, and evaluate patient 10 for medical conditions (e.g., heart failure, sleep apnea, or chronic obstructive pulmonary disease (COPD)) based on such values. The at least one parameter may include, as examples, any combination of pulse transit time or pulse wave velocity, StO₂, rSO₂, SpO₂, SvO₂, SaO₂, patient motion level, patient posture, ambient light level, optical signal quality, subcutaneous tissue impedance, heart rate, heart rate variability, respiration rate, respiration volume, and temperature.

The one or more optical sensors may include one or more light emitters and one or more light detectors. In some examples, an optical emitter of medical device 30 may output light to tissue of patient 10. A first optical detector and a second optical detector may receive the light reflected from the tissue of patient 30. The optical detectors and optical emitter may be located near a blood vessel, e.g., 20, carrying blood from heart 40, i.e., near an artery or arteriole. Processing circuitry of system 80, such as processing circuitry located within a housing of medical device 30, may receive an indication from the first optical detector of a pressure wave from a cardiac contraction passing the first optical detector and after some time delay, may receive an indication from the second optical detector of the pressure wave from the cardiac contraction passing the second optical detector. Based on the indications from the optical detectors, processing circuitry of system 80 may determine a PTT of the pressure wave between the first optical detector and the second optical detector. The processing circuitry may further calculate a pulse wave velocity based on the distance between the first optical detector and the second optical detector.

In some examples, based on the measured PTT, processing circuitry for medical device 30 may determine a change in PTT that indicates an urgent condition for patient 10. Medical device 30 may output an electronic signal to external computing device 50, or similar computing device of system 80 to alert patient 10, or a caregiver of a possible urgent condition. Some examples of an urgent condition may include a change in PTT above a threshold change amount, fluctuations in PTT or some similar condition.

In other examples, medical device 30 may include one or more other functions along with the PTT function, such as a glucose monitor, a drug pump, a device configured to manage cardiac rhythm such as an implantable pulse generator or implantable cardiac defibrillator, and other implantable medical devices. For external medical equipment, the PTT function may be integrated into an oximeter, blood pressure monitor, or a Holter monitor. For wearable, it can be integrated into a smart watch. In some examples, changes in PTT may be combined with indications from other functions of implantable, or non-implantable devices, to generate a message to an external computing device to alert the patient or caregiver.

The one or more light emitters of medical device 30 may, in some cases, consume more energy than the one or more light detectors. Additionally, a wearable or implantable medical device may be powered by an electrical energy storage component (e.g., a battery, a capacitor or similar energy storage component) that may be equipped with a limited amount of charge. For these reasons, among other reasons, it may be beneficial to limit an amount of time that the light emitters are activated to preserve device longevity.

In this manner, medical device 30 may provide advantages over other types of devices, including devices with a multidimensional optical sensor that is configured to generate images. When compared to the photo detector approach of medical device 30 of this disclosure, such a complex optical sensor would require a much more complex hardware and more power consumption, along with speed and complex circuits required for image processing. In contrast, medical device 30 of system 80 may provide a smaller, less complex, lower cost, less obtrusive solution to measuring some hemodynamic characteristics of patient 10, such as pulse wave velocity.

FIG. 1B is a conceptual diagram illustrating an example medical device arranged in proximity to tissue of the patient. Medical device 70 is an example of medical device 30 depicted in FIG. 1A and may have the same functions and characteristics. As described above in relation to FIG. 1A medical device 70 may be implanted near target tissue of the patient, e.g., blood vessel 84 in some examples. In other examples, medical device 70 may be located proximate to the skin of the patient where blood vessel 84 is in the field of light for the optical emitters and optical sensors of medical device 70 and may be held in place with a strap, adhesive or some similar mechanism.

Medical device 70 may include one or more light emitter(s) 75, with a light source, such as an LED, that may emit light at one or more wavelengths within the visible (VIS) and/or near-infrared (NIR) spectra. For example, light emitter(s) 75 may emit light at one or more wavelengths of about 590 nanometer (nm), 660 nm, 720 nm, 760 nm, 800 nm, or at any other suitable wavelengths. Medical device 70 may output light to tissue of the patient using light emitter(s) 75 to emit light at one or more VIS wavelengths, one more NIR wavelengths, or a combination of one or more VIS wavelengths and one more NIR wavelengths.

In the example of FIG. 1B, light detectors 74, 76 each may receive light from light emitter(s) 75 that is reflected by the tissue, and generate electrical signals indicating the intensities of the light detected by light detectors 74, 76. Processing circuitry of medical device 70 then may evaluate the electrical signals from light detectors 74 and 76. As shown in FIG. 1B, the first optical detector, e.g., light detector 74, and the second optical detector, light detector 76 may define a vector 72 between the first optical detector and the second optical detector with a specified length. In some examples the length of vector 72 may be less than 10 mm or longer than 10 mm.

As described above in relation to FIG. 1A, vector 72 may not necessarily align with a longitudinal axis 78 of blood vessel 84 for medical device 70 to determine a pulse transit time for a pulse wave traveling through blood vessel 84. Medical device 70 may determine the PTT while blood vessel 84 is in the field of light of and the longitudinal axis 78 does not run perpendicular to vector 72 defined by the two photo detectors.

As described above in relation to FIG. 1A, the device processing circuitry may calculate a pulse wave velocity based on the distance between the first optical detector and the second optical detector. Providing an output value for the patient, or caregiver, for PWV may be desirable to compare the PWV value for a particular patient to a typical, normal and abnormal values of PWV collected from a population of patients. In some examples, medical device 70 may be configured to determine a value of PWV based on the distance the pressure wave travels in blood vessel 84. Medical device 70, and/or other processing circuitry described above in relation to FIG. 1A, may execute a calibration process to determine the relationship between vector 72 and the length of blood vessel 84 measured by light detectors 74 and 76. For example, the operation of medical device 30 may be compared to a temporary, external PWV device, which is configured to measure the PWV over a longer distance along blood vessel 20. The simultaneous measurements between the external PMV device and medical device 30 may be retained, e.g., at a memory location in medical device 30 or elsewhere in system 80, to allow the relative PWV measured by the device to give an accurate indication of PWV.

In other examples, medical device 70 may be configured to track the relative changes in PWV over time to provide information regarding the changes in rigidity of blood vessel 84, e.g., because of increasing arteriosclerosis, or other conditions. To track changes over time, a perfect alignment of device 70 to blood vessel 84 as well as the calibration process above may not be needed.

In some examples, light emitter(s) 75 may include an optical filter between light emitter(s) 75 and an insulative cover of medical device 30 (not shown in FIG. 1B), which may limit the spectrum of emitted light to be within a narrow band. Similarly, light detectors 74 and 76 may include optical filters between light detectors 74 and 76 and the insulative cover so that light detectors 74 and 76 detects light from a narrow spectrum, which may have longer wavelengths than the emitted spectrum. Other optical elements that may be included in the medical device 70 may include index matching layers, antireflective coatings, or optical barriers, which may be configured to block light emitted sideways by the light emitter(s) 75 from reaching light detectors 74 and 76. In some examples, optical filters and index matching layers may also reduce the impact of ambient light on the light detectors.

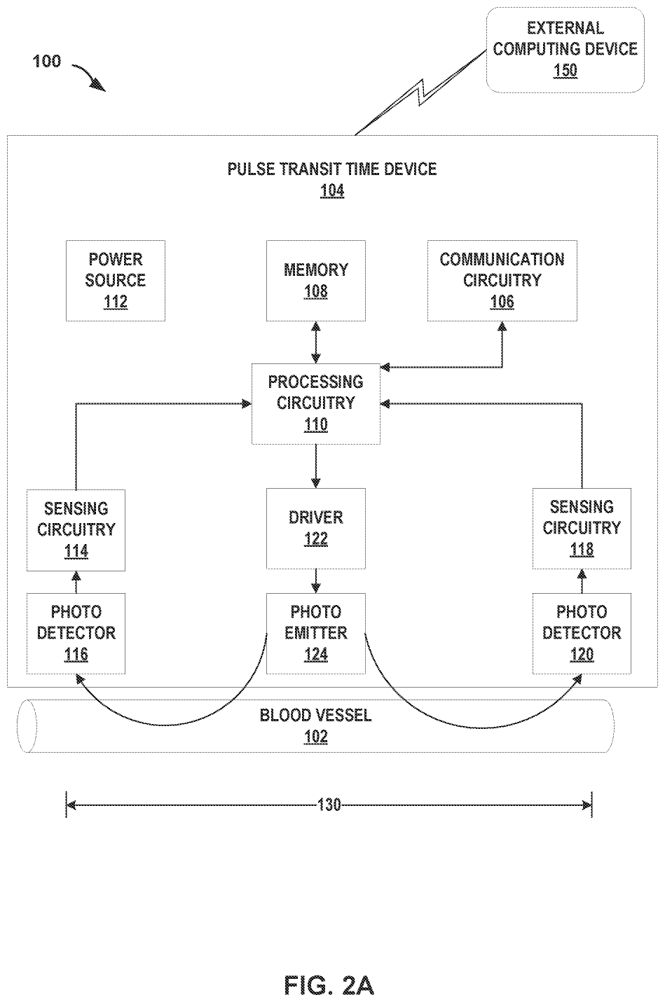

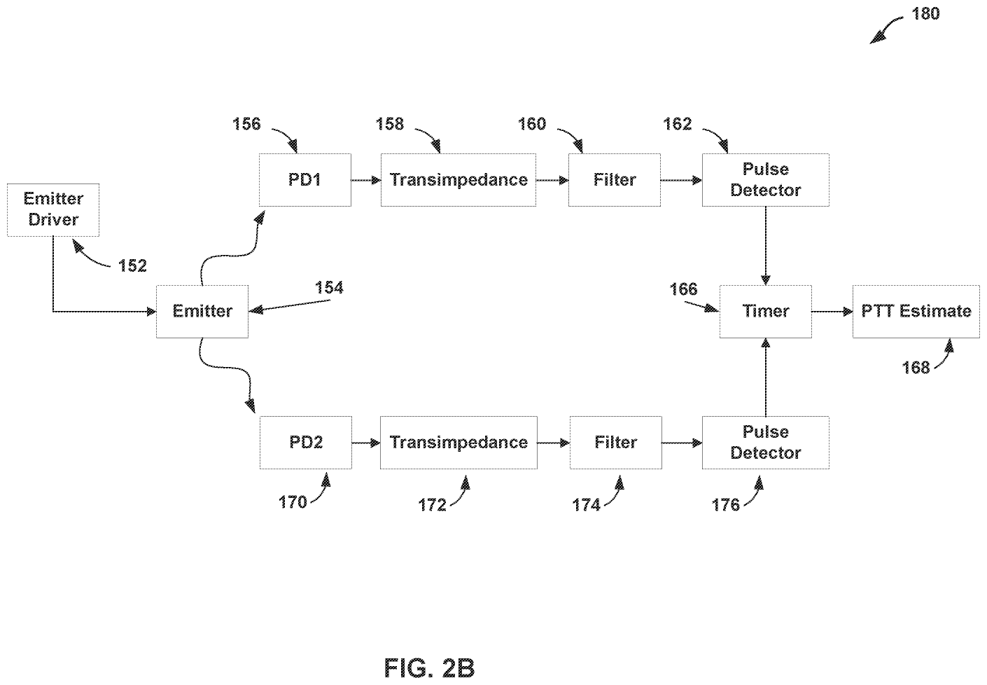

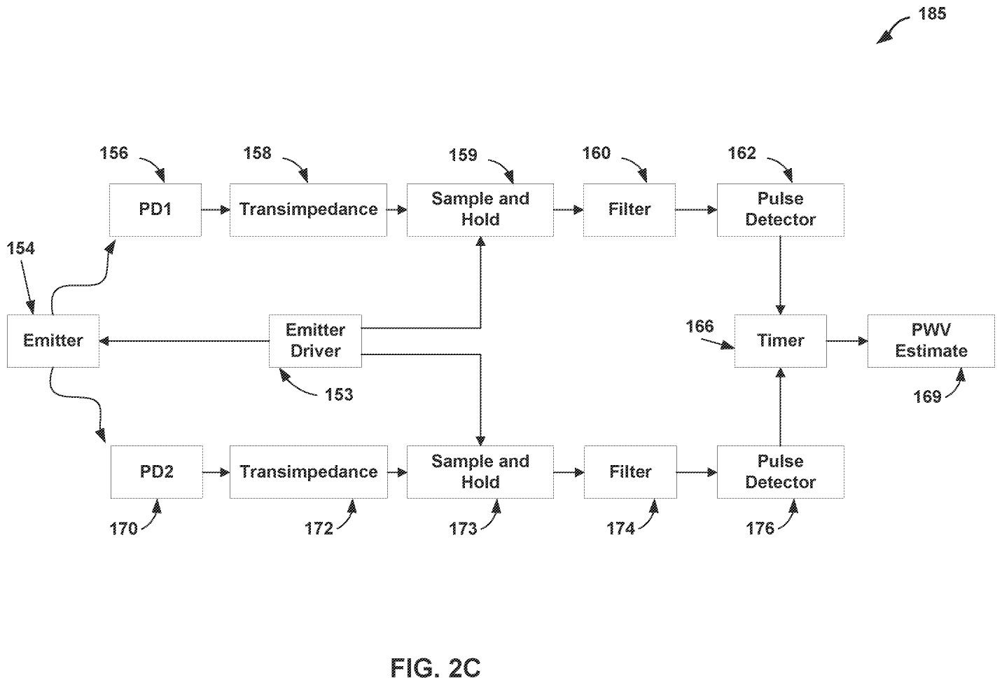

FIG. 2A is a block diagram illustrating an example implementation of device configured to measure pulse transit time, which uses a single optical emitter. System 100 is an example of system 80 described above in relation to FIG. 1A and the components of system 100 have the same functions and characteristics as the components of system 80. For example, external computing device 150 and pulse transit time device 104 are examples, respectively, of external computing device 50 and medical device 30 depicted in FIG. 1A. Pulse transit time device 104 (PTT device 104) is also an example of medical device 70 depicted in FIG. 1B.

In the example of FIG. 2A, system 100 includes external computing device 150, and PPT device 104. PTT device 104 includes photo emitter 124, processing circuitry 110, sensing circuitry 114 and 118, communication circuitry 106, memory 108, one or more sensors including photo detectors 116, and power source 112. Processing circuitry 110 may control the operation of photo emitter 124 via driver 122, and receive an indication from photo detector 116 and photo detector 120 of a pressure wave from a cardiac contraction passing photo detector 116 and photo detector 120. Sensing circuitry 114 and 118 may include amplifiers, filters and other circuitry to conduct signals from photo detector 116 and photo detector 120 to processing circuitry 110.

Examples of processing circuitry 110 may include any one or more of a microcontroller (MCU), e.g. a computer on a single integrated circuit containing a processor core, memory, and programmable input/output peripherals, a microprocessor (μP), e.g. a central processing unit (CPU) on a single integrated circuit (IC), a controller, a digital signal processor (DSP), an application specific integrated circuit (ASIC), a field-programmable gate array (FPGA), a system on chip (SoC) or equivalent discrete or integrated logic circuitry. A processor may be integrated circuitry, i.e., integrated processing circuitry, and that the integrated processing circuitry may be realized as fixed hardware processing circuitry, programmable processing circuitry and/or a combination of both fixed and programmable processing circuitry. Accordingly, the terms “processing circuitry,” “processor” or “controller,” as used herein, may refer to any one or more of the foregoing structures or any other structure operable to perform techniques described herein. Functions attributed to processing circuitry 110 may in some examples be shared between external computing device 150 and processing circuitry 110.

Examples of memory 108 may include any type of computer-readable storage media. include random access memory (RAM), read only memory (ROM), programmable read only memory (PROM), erasable programmable read only memory (EPROM), one-time programable (OTP) memory, electronically erasable programmable read only memory (EEPROM), flash memory, or another type of volatile or non-volatile memory device. In some examples the computer readable storage media may store instructions that cause the processing circuitry to execute the functions described herein. In some examples, the computer readable storage media may store data, such as configuration information, temporary values and other types of data used to perform the functions of this disclosure.

Communication circuitry 106 may include any suitable hardware, firmware, software or any combination thereof for communicating with another device, such as external device 150 or another IMD or sensor, such as a pressure sensing or continuous glucose monitoring device. Under the control of processing circuitry 110, communication circuitry 106 may receive downlink telemetry from, as well as send uplink telemetry to, external device 150 or another device with the aid of an internal or external antenna (not shown in FIG. 2A). In addition, processing circuitry 106 may communicate with a networked computing device, e.g., servers 60 depicted in FIG. 1A, via external device (e.g., external device 150) and a computer network, such as the Medtronic CareLink® Network developed by Medtronic.

Communication circuitry 106 may be configured to transmit to or receive signals via inductive coupling, electromagnetic coupling, tissue conductance, NFC, Radio Frequency Identification (RFID), Bluetooth®, WiFi, or other proprietary or non-proprietary wireless telemetry communication schemes. In the example in which PTT device 104 is a wearable or portable device, communication circuitry 106 may also operate using wired communication. A clinician or other user may retrieve data from PTT device 104 using external device 150, or by using another local or networked computing device configured to communicate with processing circuitry 110 via communication circuitry 106. The clinician may also program parameters of PTT device 104 using external device 150 or another local or networked computing device.

Power source 112 may be configured to deliver operating power to the components of PTT device 104. Power source 112 may include a capacitor, a battery, including a rechargeable battery, or similar electrical energy storage component as well as a power generation circuit to produce the operating power. In some examples, the battery is rechargeable to allow extended operation. In some examples, recharging is accomplished through proximal inductive interaction between an external charger and an inductive charging coil within external device 150. Power source 112 may include any one or more of a plurality of different battery types, such as nickel cadmium batteries and lithium-ion batteries. A non-rechargeable battery may be selected to last for several years, while a rechargeable battery may be inductively charged from an external device, e.g., on a daily, weekly or monthly basis.

In operation, processing circuitry 110 of PTT device 104 may control photo emitter 124 to output light to blood vessel 102. Processing circuitry 110 may be configured to control driver 122 to cause photo emitter 124 to operate in either a continuous mode or a pulsed mode. In some examples, pulsed mode may reduce energy consumption when compared to continuous mode.

Processing circuitry 110 may receive an indication from the photo detector 116, via sensing circuitry 114 of a pressure wave from a cardiac contraction passing photo detector 116. Processing circuitry 110 may further receive an indication from the photo detector 120 of the pressure wave from the cardiac contraction passing photo detector 120. Processing circuitry 110 may include timer configured for a temporal resolution that enables measurement of PTT as described herein, for example, to detect a change of less than or equal to 100 microseconds (μsec) in pulse transit times during a monitoring period, and may determine a pulse transit time of the pressure wave between the photo detector 116 and the photo detector 120.

As described above in relation to FIG. 1B, photo detector 116 and photo detector 120 may define a vector of length 130. In the example of FIG. 2A, the distance between photo emitter 124 and each photo detector may be different from the distance (130) between the two photo detectors.

Because the current drain for photo emitter 124 dominates the overall current drain of the system, in some examples, processing circuitry 110 may control photo emitter 124 to operate with a duty cycle to reduce the power consumption and improve the longevity of power source 112. Therefore, in some examples, photo emitter 124 may operate for only a few seconds a day. In some examples system 100 may be configured to measure PTT one or more times per day. In some examples, the drive current for the LED or VCSEL is around 5 mA. As described above in relation to FIG. 1B, in some examples, system 100 may be configured to track changes in PTT, and calculate changes in PWV, which may monitor for changes in the rigidity of blood vessel 102. In this manner, system 100 may need only take periodic, e.g., daily or fewer measurements, to monitor PTT for the patient.

In examples in which electro-cardiogram (ECG) sensing is available system 100 may be configured to further reduce duty cycle the on-time for the PTT channel by gating the PTT measurement ON at a cardiac sensed event and then turning the PTT channel off after a preset amount of time. In some examples, device 104 may also include electrodes and sensing circuitry (not shown in FIG. 2A) to sense cardiac events. In other examples some other device implanted or worn by the patient may provide ECG sensing and communicate with system 100. This duration of the on-time for the PTT channel of device 104 may be based on an expected delay from the electrically sensed cardiac event to the optical PTT pulse. This delay depends on a number of factors, such as implant location. In some examples, by coordinating the duty cycle of the PTT channel based on the ECG sensed event, device 104 may reduce current usage by, for example eight to ten times, compared to other modes of operation.

FIGS. 2B and 2C are block diagrams illustrating example pulse transit time measuring devices with a single optical emitter. PTT device 180 is an example of PTT device 104, medical device 30 and medical device 70 described above in relation to FIGS. 1A, 1B and 2A and may have the same or similar functions and characteristics. In some examples the circuitry of PTT device 180 may be implemented as an analog circuit, which may reduce size and power consumption compared to other implementations. In other examples, such as implementing device 180 as a wearable external device, rather than a miniaturized implantable device, the circuit of FIG. 2B may be realized as a digital circuit. PTT device 180 is configured to detect the passage of the pulse wave and measure the time between the passage of the pulse wave for each detector.

In the example of FIG. 2B, photo detector (PD 1) 156 outputs a signal to transimpedance amplifier 158. Transimpedance amplifier receives the electrical current signal from photo detector 156 and outputs a representative amplified voltage signal to filter 160. Filter 160 receives and filters the amplified signal and outputs the filtered signal to pulse detector 162. In some examples, filter 160 may include a band-pass filter to remove high frequency noise and to establish a stable baseline.

In some examples, PTT device 180 may operate in either a continuous mode or in a pulsed mode e.g., to conserve power. Operating in a pulsed mode, a battery powered device may last longer before replacement, or longer between recharging the battery. In a pulsed mode, the circuitry of a PTT device 180 may include sample and hold circuitry 159 and 173, which is shown in FIG. 2C for PTT device 185 (not shown in FIG. 2B). Sample and hold circuitry may be desirable, but not necessarily required, for pulsed mode operation.

When operating in pulsed mode, the circuitry shown in either FIGS. 2B or 2C may use filters 160 and 174 to recover the desired signal from the sampled signal. In some examples, filters 160 and 174 may be implemented as low pass filters. In the example of FIG. 2C filters 160 and 174 are at the output of the sample and hold 159 and 173 respectively. The low pass filters are intended to provide a reconstructed continuous signal from the sampled signal. The intent is to recover the relative PTT timing from the difference in reconstructed signal peaks from PD1 & PD2. In some examples it may be desirable to constrain the low pass filter in three ways: 1) the filters on both pathways should be matched. Matching the filters means that the time delay through each pathway will be matched; 2) the filter bandwidth should be low enough to suppress the sampling frequency, e.g., 1 kHz or some other sampling frequency. This constraint may be useful in resolving, e.g., a 100 us delay using a 1 KHz sampling; and 3) the bandwidth should be high enough to not distort the optical waveform from the PD, e.g., PD 156 and 170. If the bandwidth is too low, some timing information could be lost making the PTT measurement less accurate.

In some examples of pulsed operation, such as shown in FIG. 2C, emitter driver 153 may turn on the emitter 154 briefly and turn it off at a specific frequency and duty cycle. The frequency that the emitter is turned on may be 1 kHz or higher. As noted above, operating in pulsed mode may reduce the power required for the intended measurement. When operating in pulsed mode, emitter driver 153 may also output timing signals, also called control signals, to sample and hold circuitry 159 and 173 for holding the signal at its last sampled level, which may also reduce high frequency noise caused from the turning-off of the emitter 154.

In some examples, as noted above, the circuitry of this disclosure may be configured to detect small changes in the travel time for the pressure wave, such as changes on the order of 100 μs or less. In pulsed mode, emitter 154 may operate at a frequency to resolve 100 μs sample frequency and therefore emitter driver 152, or 153, may drive emitter 154 at a frequency of up to 10 kHz, or higher. In other examples, such as operating when low power consumption is desirable, emitter drivers 152 or 153 may operate at frequencies in the range of 5 kHz to 20 kHz. In other examples, such as when detecting small changes is desirable, emitter drivers 152 or 153 may operate to drive emitter 154 in the range of 10 kHz to 50 kHz or higher, e.g., 100 kHz. In other examples, to both reduce power consumption and maintain high resolution, emitter drivers 152 or 153 may operate in bursts of 10 kHz, or higher, for a first period of time, e.g., a few seconds, and operated at a lower frequency, such as 1 kHz to 15 kHz during a second period of time.

Pulse detector 162 may be configured to detect the passage of the pulse wave in several different ways. In some examples, pulse detector 162 may include circuitry to detect the peak, or a valley in the optical signal that indicates the passage of the pulse wave. In other examples, pulse detector 162 include circuitry configured to determine a derivative of the received indication from photo detector 156. Taking the derivative of this pressure wave signal results in a signal that is proportional to the slope of the pressure wave. At the peak of the pressure wave, the slope is zero. Similarly at the lowest point in a valley of the signal, the slope is zero. Correspondingly, the derivative signal will be at zero at the pressure wave peak. In some examples pulse detector 162 may include circuitry to detect the zero crossing of the derivative signal, which will correspond to the peak of the pressure wave.

Implementing pulse detector 162 with a differentiator circuit and zero crossing detect circuit is one example implementation. In other examples, pulse detector 162 may implemented using other circuitry such as to detect the region of greatest change, e.g., the portion of the signal with the fastest slew rate or highest change per unit time, e.g., dv/dt.

Similarly, photo detector (PD 2) 170 outputs a signal to transimpedance amplifier 172. Transimpedance amplifier receives the electrical current signal from photo detector 170 and outputs a representative amplified voltage signal to filter 174. Filter 174 receives and filters the amplified signal and outputs the filtered signal to pulse detector 176. Pulse detector 176 may provide an output to timer 166 that a pressure wave passed photo detector 170.

Timer 166, which may be included in the processing circuitry of PTT device 180, may determine the pulse transit time based on any difference between the time in the output from pulse detector 162 and pulse detector 172. The processing circuitry may then calculate the PTT estimate 168, and in some examples, also calculate a pulse wave velocity estimate 169 as shown in FIG. 2C. Emitter driver 152 may receive signals from the processing circuitry to cause photo emitter 154 to output light to tissue of the patient.

The functions listed in FIG. 2B may be grouped under either sensing circuitry or processing circuitry. For example, transimpedance amplifiers 158 and 172 and filters 160 and 174 may be considered sensing circuitry, e.g., part of sensing circuitry 114 or 118 respectively, while other functions, e.g., timer 166 may be handled by processing circuitry 110. In other examples, processing circuitry may perform timing 166 and PTT estimate 168, while sensing circuitry may perform the other functions. Some arrangements may consume less power than other arrangements, for example, implementing filter 174, pulse detector 176 as hardware circuits may allow a processor to be in low power or sleep mode for more time and may reduce power consumption of the circuit.

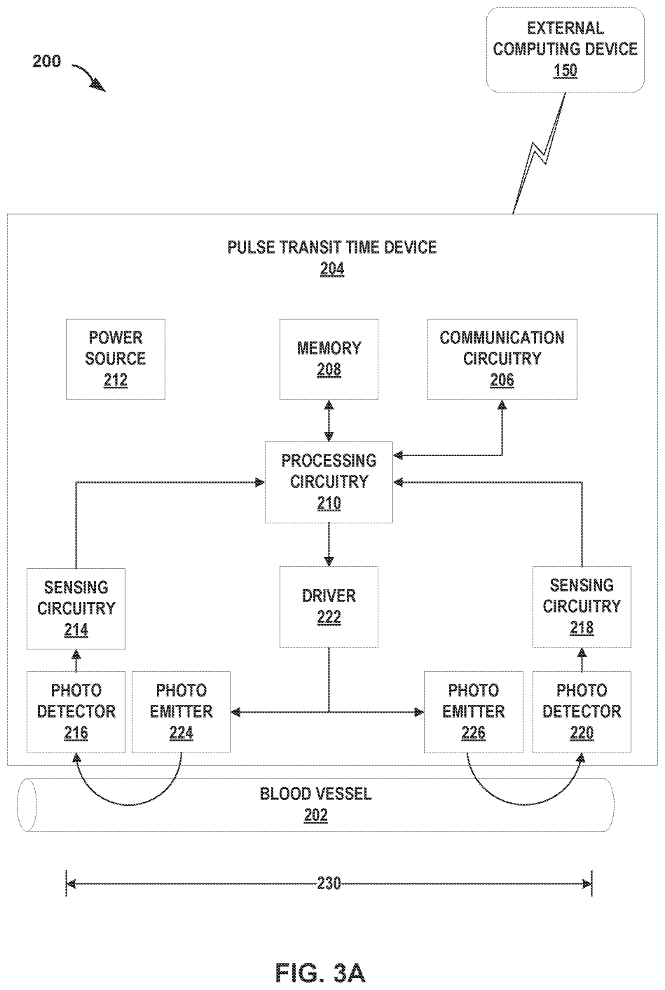

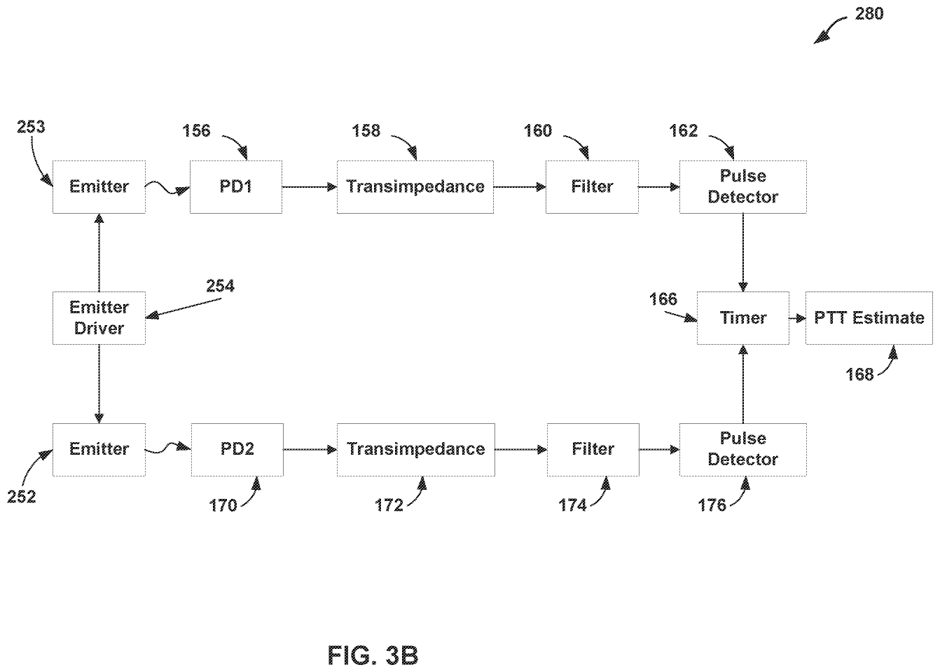

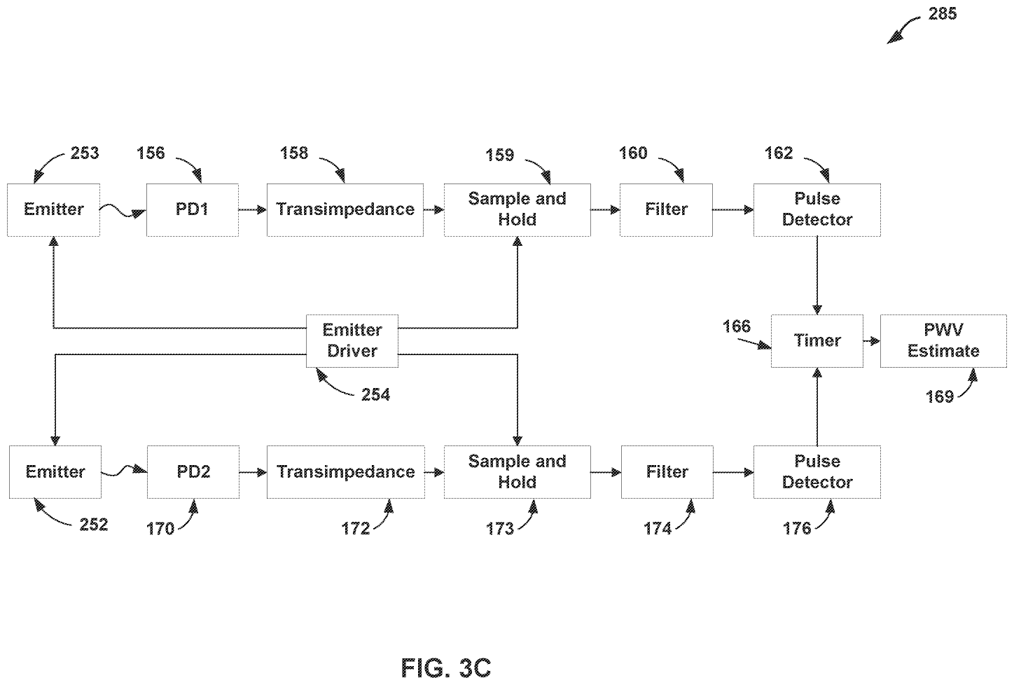

FIG. 3A is a block diagram illustrating an example implementation of device configured to measure pulse transit time, which uses at least two optical emitters. System 200 is an example of system 80 described above in relation to FIG. 1A and the components of system 100 have the same functions and characteristics as the components of system 80. PTT device 204 is an example of medical device 30 and medical device 70 described above in relation to FIGS. 1A, 1B as well as an example of PTT device 104, and PTT device 180 depicted in FIGS. 2A and 2B and have the same or similar functions and characteristics, with the exception that emitter driver 222 of system 200 drives two photo emitters 224 and 226, where emitter driver 122 of system 100 drives a single photo emitter 124. Otherwise, the functions and characteristics described above for system 100 apply equally to system 200.

Specifically, in the example of FIG. 3A, system 200 includes external computing device 150 and PPT device 204. PTT device 204 includes photo emitter 224, processing circuitry 110, sensing circuitry 114 and 118, communication circuitry 106, memory 208, one or more sensors including photo detectors 216, and power source 212. Processing circuitry 210 may control the operation of photo emitter 224 and photo emitter 226 via driver 222 and receive an indication from photo detector 216 and photo detector 220 of a pressure wave from a cardiac contraction passing photo detector 216 and photo detector 220. As described above for FIGS. 2A and 2B, sensing circuitry 214 and 218 may include amplifiers, filters and other circuitry to conduct signals from photo detector 216 and photo detector 220 to processing circuitry 210.

Communication circuitry 206 may include any suitable hardware, firmware, software or any combination thereof for communicating with another device. Under the control of processing circuitry 210, communication circuitry 106 may receive downlink telemetry from, as well as send uplink telemetry to for example external device 150. Communication circuitry 206 may be configured to transmit to or receive signals via inductive coupling, electromagnetic coupling, tissue conductance, NFC, Radio Frequency Identification (RFID), Bluetooth®, WiFi, or other proprietary or non-proprietary wireless telemetry communication schemes.

Power source 212 may be configured to deliver operating power to the components of PTT device 204. Power source 212 may include a capacitor, a battery or similar electrical energy storage component as well as a power generation circuit to produce the operating power.

Similar to what was described above for systems 100 and 180, in operation, processing circuitry 210 of PTT device 204 may control photo emitters 224 and 226 to output light to blood vessel 202. In the example of FIG. 3A, photo detector 216 is configured to receive the light from photo emitter 224 reflected from the tissue of the patient, while photo detector 220 is configured to receive the light from photo emitter 226 reflected from the tissue of the patient.

Processing circuitry 210 may receive an indication from the photo detector 216, via sensing circuitry 214 of a pressure wave from a cardiac contraction passing photo detector 216. Processing circuitry 210 may further receive an indication from the photo detector 220 of the pressure wave from the cardiac contraction passing photo detector 220. As with the devices described above in relation to FIGS. 1A-2C, processing circuitry 210 may include one or more timers configured for a high temporal resolution, for example, such that processing circuitry 210 may detect changes between pulse transit times, over the duration of a monitoring period, of less than or equal to 100 microseconds (μsec) and may determine a pulse transit time of the pressure wave between the photo detector 216 and the photo detector 220. In some examples, the timer resolution may be less than 10 μsec to detect differences of less than or equal to 100 μsec between pulse transit times. In other examples, the timer resolution may be based on the operating frequency, as described above in relation to FIGS. 2B and 2C.

As described above in relation to FIG. 1B, photo detector 216 and photo detector 220 may define a vector of length 230. In the example of FIG. 3A, the distance between photo emitter 224 and photo detector 216 may be less than the distance (230) between the two photo detectors.

FIG. 3B is a block diagram illustrating an example pulse transit time measuring device with two or more optical emitters. PTT device 280 is an example of PTT device 204 described above in relation to FIG. 3A. PTT device 280 is also an example of medical device 30 and medical device 70 described above in relation to FIGS. 1A, 1B as well as an example of PTT device 104, and PTT device 180 depicted in FIGS. 2A and 2B and have the same or similar functions and characteristics, with the exception that emitter driver 254 of PTT device 280 drives two photo emitters 252 and 253. Otherwise, the functions and characteristics described above for PTT device 180 apply equally to PTT device 280.

Similar to FIG. 2C, the example of FIG. 3C illustrates PTT device 285 that includes sample and hold circuitry 159 and 173. As described above in relation to FIGS. 2B and 2C, PTT device 280, or any other device of this disclosure, may operate in either a continuous mode, or in a pulsed mode, which may conserve power when compared to the continuous mode. As with emitter driver 152 and 153, in pulsed mode emitter driver 254 may output a timing signal to sample and hold circuitry 159 and 173, in addition to driving emitter 252 and emitter 253. Also, as with the examples of FIGS. 2B and 2C, in some examples the circuitry of FIGS. 3B and 3C may output PTT estimate 168, while in other examples, the circuitry may output PWV estimate 169.

As described above for FIGS. 2B and 2C, the example of FIG. 3C, the emitter driver 254 may turn on the emitters 252 and 253 briefly and turns it off at a specific frequency and duty cycle when in pulsed mode. The frequency that the emitters are turned on can be 1 kHz or higher, or operate in any of the frequency ranges described above, which may reduce the power required for the intended measurement. The timing signal from emitter driver 254 to sample and hold circuitry 159 and 173 may also reduce the high frequency noise (e.g., ringing) caused by turning-off of emitters 252 and 253.

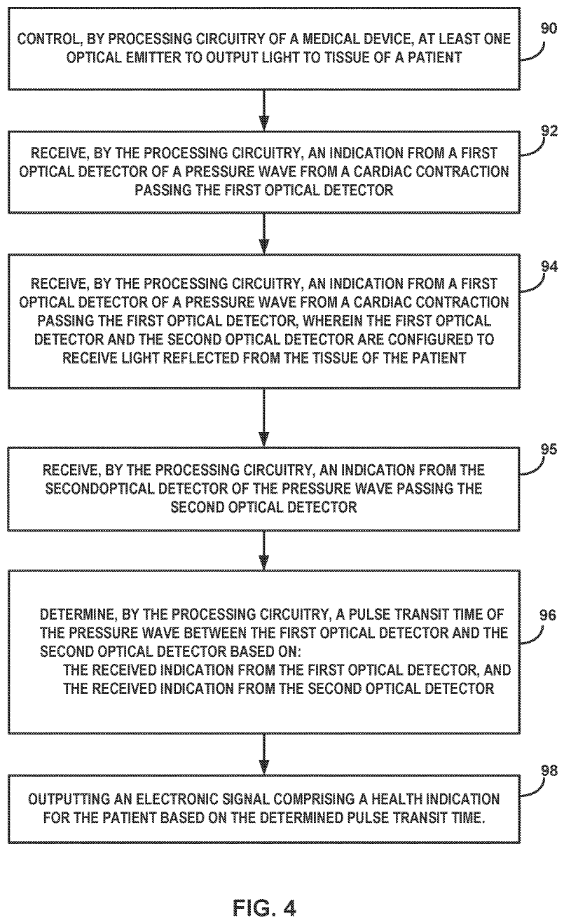

FIG. 4 is a flow chart illustrating an example operation of the pulse transit time measurement system of this disclosure. As seen in the example of FIG. 4, processing circuitry, e.g., processing circuitry 54 described above in relation to FIG. 1, may initially control at least one optical emitter, e.g., photo emitter 124 of FIG. 2A, to output light to tissue of a patient, e.g., blood vessel 102 of FIG. 2A (90). Next, processing circuitry 54 may receive an indication from a first optical detector, e.g., photo detector 116 described above in relation to FIG. 2A, of a pressure wave from a cardiac contraction passing the first optical detector (92).

Processing circuitry, such as processing circuitry 110 of FIG. 2A may also receive an indication from the first optical detector of a pressure wave from a cardiac contraction passing first optical detector 116 based on light from photo emitter 124 reflected from the tissue of patient 10, e.g., from blood vessel 102 (94). Processing circuitry 110 may further receive an indication from the second optical detector, e.g., photo detector 120 of FIG. 2A, of the pressure wave from the cardiac contraction passing the second optical detector (95).

Processing circuitry 110 may determine a pulse transit time of the pressure wave between the first optical detector and the second optical detector based on the received indication from the first optical detector, and the received indication from the second optical detector, as described above in relation to FIG. 2B (96). In some examples, the indication may include a derivative of the received indication from the first optical detector, and a derivative of the received indication from the second optical detector, as well as a zero crossing indication. The processing circuitry may further output an electronic signal comprising a health indication for the patient based on the determined pulse transit time, as described above in relation to FIG. 1 (98).

The techniques of this disclosure may also be described in the following examples.

Example 1: A medical system comprises: a medical device including: at least one optical emitter configured to output light to tissue of a patient; a first optical detector and a second optical detector configured to receive the light reflected from the tissue of the patient; processing circuitry configured to: receive an indication from the first optical detector of a pressure wave from a cardiac contraction passing the first optical detector; receive an indication from the second optical detector of the pressure wave from the cardiac contraction passing the second optical detector; determine a pulse transit time of the pressure wave between the first optical detector and the second optical detector based on: the received indication from the first optical detector, and the received indication from the second optical detector; and output an electronic signal comprising a health indication for the patient based on the determined pulse transit time.

Example 2: The system of example 1, wherein the at least one optical emitter comprises a first optical emitter and a second optical emitter; wherein the first optical detector is configured to receive the light from the first optical emitter reflected from the tissue of the patient, and wherein the second optical detector is configured to receive the light from the second optical emitter reflected from the tissue of the patient.

Example 3: The system of any of examples 1 and 2, further comprising a timer configured for a temporal resolution to detect changes of less than or equal to 100 microseconds (μsec) between pulse transit times, during a monitoring period.

Example 4: The system of any of examples 1 through 3, wherein a distance between the first optical detector and the second optical detector is less than or equal to fifty millimeters (mm).

Example 5: The system of any of examples 1 through 4, wherein the medical device is configured to be implanted in the patient.

Example 6: The system of any of examples 1 through 5, wherein the processing circuitry is further configured to calculate a pulse wave velocity for the pressure wave based on the pulse transit time.

Example 7: The system of any of examples 1 through 6, wherein the medical device comprises the processing circuitry.

Example 8: The system of any of examples 1 through 7, wherein the first optical detector and the second optical detector define a vector between the first optical detector and the second optical detector wherein the medical device is arranged relative to a blood vessel carrying the pressure wave: wherein the blood vessel is located within a field of the light output by the optical emitter, and wherein the vector is other than perpendicular to a longitudinal axis of the blood vessel.

Example 9: The system of any of examples 1 through 8, wherein the medical device comprises an energy storage component configured to provide power to components of the medical device, and wherein the energy storage component is a battery.

Example 10: A medical device comprises: at least one optical emitter configured to output light to tissue of a patient; a first optical detector and a second optical detector configured to receive the light reflected from the tissue of the patient; processing circuitry configured to: receive an indication from the first optical detector of a pressure wave from a cardiac contraction passing the first optical detector; receive an indication from the second optical detector of the pressure wave from the cardiac contraction passing the second optical detector; determine a pulse transit time of the pressure wave between the first optical detector and the second optical detector based on: the received indication from the first optical detector, and the received indication from the second optical detector; and output an electronic signal comprising a health indication for the patient based on the determined pulse transit time.

Example 11: The medical device of example 10, wherein the at least one optical emitter comprises a first optical emitter and a second optical emitter; wherein the first optical detector is configured to receive the light from the first optical emitter reflected from the tissue of the patient, and wherein the second optical detector is configured to receive the light from the second optical emitter reflected from the tissue of the patient.

Example 12: The medical device of any of examples 10 and 11, further comprising a timer configured to detect changes of less than or equal to 100 microseconds (μsec) between pulse transit times.

Example 13: The medical device of any of examples 10 through 12, wherein a distance between the first optical detector and the second optical detector is less than or equal to fifty millimeters (mm).

Example 14: The medical device of any of examples 10 through 13, wherein the medical device is configured to be implanted in the patient.

Example 15: The medical device of any of examples 10 through 14, wherein the medical device is configured to be worn by the patient.

Example 16: The medical device of any of examples 10 through 15, wherein the processing circuitry is further configured to calculate a pulse wave velocity for the pressure wave based on the pulse transit time.

Example 17: The medical device of any of examples 10 through 16, wherein the first optical detector and the second optical detector define a vector between the first optical detector and the second optical detector wherein the medical device is arranged relative to a blood vessel carrying the pressure wave: wherein the blood vessel is located within a field of the light output by the optical emitter wherein the vector is other than perpendicular to a longitudinal axis of the blood vessel.

Example 18: The medical device of any of examples 10 through 16, wherein the processing circuitry is configured to determine a pulse transit time of the pressure wave between the first optical detector and the second optical detector based on: a derivative of the received indication from the first optical detector, and a derivative of the received indication from the second optical detector.

Example 19: A method comprising: controlling, by processing circuitry of a medical device, at least one optical emitter to output light to tissue of a patient; receiving, by the processing circuitry, an indication from a first optical detector of a pressure wave from a cardiac contraction passing the first optical detector; receiving, by the processing circuitry, an indication from a second optical detector of a pressure wave from a cardiac contraction passing the second optical detector, wherein the first optical detector and the second optical detector are configured to receive light reflected from the tissue of the patient; determining, by the processing circuitry, a pulse transit time of the pressure wave between the first optical detector and the second optical detector based on: the received indication from the first optical detector, and the received indication from the second optical detector; and outputting an electronic signal comprising a health indication for the patient based on the determined pulse transit time.

Example 20: The method of example 19, wherein the at least one optical emitter is a first optical emitter, the method further includes controlling, by the processing circuitry, a second optical emitter to output light to the tissue of the patient, wherein the first optical detector is configured to receive the light from the first optical emitter reflected from the tissue of the patient, and wherein the second optical detector is configured to receive the light from the second optical emitter reflected from the tissue of the patient.

Example 21: The method of any of examples 20 and 21, wherein the first optical detector and the second optical detector define a vector between the first optical detector and the second optical detector wherein the medical device is arranged relative to a blood vessel carrying the pressure wave: wherein the blood vessel is located within a field of the light output by the optical emitter, and wherein the vector is other than perpendicular to a longitudinal axis of the blood vessel.

Various examples of the disclosure have been described. These and other examples are within the scope of the following claims.

1. A system comprising:
a medical device including:
at least one optical emitter configured to output light to tissue of a patient; and
a first optical detector and a second optical detector configured to receive the light reflected from the tissue of the patient; and
processing circuitry configured to:
receive an indication from the first optical detector of a pressure wave from a cardiac contraction passing the first optical detector;
receive an indication from the second optical detector of the pressure wave from the cardiac contraction passing the second optical detector;
determine a pulse transit time of the pressure wave between the first optical detector and the second optical detector based on:
the received indication from the first optical detector, and
the received indication from the second optical detector; and
output an electronic signal comprising a health indication for the patient based on the determined pulse transit time.

2. The system of claim 1,
wherein the at least one optical emitter comprises a first optical emitter and a second optical emitter,
wherein the first optical detector is configured to receive the light from the first optical emitter reflected from the tissue of the patient, and
wherein the second optical detector is configured to receive the light from the second optical emitter reflected from the tissue of the patient.

3. The system of claim 1, wherein the medical device is configured to determine a change in pulse transit time of less than or equal to 100 microseconds (μsec).

4. The system of claim 1, wherein a distance between the first optical detector and the second optical detector is less than or equal to fifty millimeters (mm).

5. The system of claim 1, wherein the medical device is configured to be implanted in the patient.

6. The system of claim 1, wherein the processing circuitry is further configured to calculate a pulse wave velocity for the pressure wave based on the pulse transit time.

7. The system of claim 1, wherein the medical device comprises the processing circuitry.

8. The system of claim 1,
wherein the first optical detector and the second optical detector define a vector between the first optical detector and the second optical detector, and
wherein the medical device is arranged relative to a blood vessel carrying the pressure wave:
wherein the blood vessel is located within a field of the light output by the optical emitter, and
wherein the vector is other than perpendicular to a longitudinal axis of the blood vessel.

9. The system of claim 1,
wherein the medical device comprises an energy storage component configured to provide power to components of the medical device, and
wherein the energy storage component is a battery.

10. A medical device comprising:
at least one optical emitter configured to output light to tissue of a patient;
a first optical detector and a second optical detector configured to receive the light reflected from the tissue of the patient; and
processing circuitry configured to:
receive an indication from the first optical detector of a pressure wave from a cardiac contraction passing the first optical detector;
receive an indication from the second optical detector of the pressure wave from the cardiac contraction passing the second optical detector;
determine a pulse transit time of the pressure wave between the first optical detector and the second optical detector based on:
the received indication from the first optical detector, and
the received indication from the second optical detector; and
output an electronic signal comprising a health indication for the patient based on the determined pulse transit time.

11. The medical device of claim 10,
wherein the at least one optical emitter comprises a first optical emitter and a second optical emitter,
wherein the first optical detector is configured to receive the light from the first optical emitter reflected from the tissue of the patient, and
wherein the second optical detector is configured to receive the light from the second optical emitter reflected from the tissue of the patient.

12. The medical device of claim 10, wherein the processing circuitry is configured to operate the medical device in pulsed mode.

13. The medical device of claim 10, wherein a distance between the first optical detector and the second optical detector is less than or equal to fifty millimeters (mm).

14. The medical device of claim 10,
wherein the first optical detector and the second optical detector define a vector between the first optical detector and the second optical detector, and
wherein the medical device is arranged relative to a blood vessel carrying the pressure wave:
wherein the blood vessel is located within a field of the light output by the optical emitter, and
wherein the vector is other than perpendicular to a longitudinal axis of the blood vessel.

15. The medical device of claim 10, wherein the processing circuitry is configured to determine the pulse transit time of the pressure wave between the first optical detector and the second optical detector based on:
a derivative of the received indication from the first optical detector, and
a derivative of the received indication from the second optical detector.

16. The medical device of claim 10, wherein the processing circuitry is further configured to calculate a pulse wave velocity for the pressure wave based on the pulse transit time.

17. The medical device of claim 10, further comprising a timer configured to detect changes of less than or equal to 100 microseconds (μsec) between pulse transit times.

18. A method comprising:
controlling, by processing circuitry of a medical device, at least one optical emitter to output light to tissue of a patient;
receiving, by the processing circuitry, an indication from a first optical detector of a pressure wave from a cardiac contraction passing the first optical detector;
receiving, by the processing circuitry, an indication from a second optical detector of the pressure wave from the cardiac contraction passing the second optical detector, wherein the first optical detector and the second optical detector are configured to receive the light reflected from the tissue of the patient;
determining, by the processing circuitry, a pulse transit time of the pressure wave between the first optical detector and the second optical detector based on:
the received indication from the first optical detector, and
the received indication from the second optical detector; and
outputting, by the processing circuitry, an electronic signal comprising a health indication for the patient based on the determined pulse transit time.

19. The method of claim 18,
wherein the at least one optical emitter is a first optical emitter,
wherein the method further includes controlling, by the processing circuitry, a second optical emitter to output light to the tissue of the patient,
wherein the first optical detector is configured to receive the light from the first optical emitter reflected from the tissue of the patient, and
wherein the second optical detector is configured to receive the light from the second optical emitter reflected from the tissue of the patient.

20. The method of claim 18,
wherein the first optical detector and the second optical detector define a vector between the first optical detector and the second optical detector, and
wherein the medical device is arranged relative to a blood vessel carrying the pressure wave:
wherein the blood vessel is located within a field of the light output by the optical emitter, and
wherein the vector is other than perpendicular to a longitudinal axis of the blood vessel.
